(19) United States
(12) Patent Application Publication
(10) Pub. No.: US 2015/0164323 A1
(43) Pub. Date: Jun. 18, 2015

(54) SECURE COMMUNICATIONS BETWEEN ELEMENTS IN A WIRELESS NETWORK

(71) Applicant: Medtronic MiniMed, Inc., Northridge, CA (US)

(72) Inventor: Kris R. Holtzclaw, Santa Clarita, CA (US)

(73) Assignee: MEDTRONIC MINIMED, INC., Northridge, CA (US)

(21) Appl. No.: 14/107,872

(22) Filed: Dec. 16, 2013

(51) Int. Cl. A61B 5/00; A61M 5/172; H04Q 9/00; A61B 5/145

(52) U.S. Cl. CPC A61B 5/0022; A61B 5/14532; A61B 5/4839; A61M 5/1723; H04Q 9/00

(57) ABSTRACT

A patient monitor/infusion system is disclosed. The monitor/infusion system includes a medical device having a first machine parsable code and a medical device transmitter, configured to broadcast encrypted data indicative of a characteristic of the user. The monitor/infusion system further includes a mobile device with a plurality of sensors to capture the first machine parsable code. The mobile device further having a receiver to receive encrypted data broadcast by the medical device and at least one of the plurality of sensors enables out-of-band pairing between the mobile device and the medical device via the first machine parsable code.

### FIELD OF THE INVENTION

This invention relates to secure wireless communication and in particular embodiments, methods and devices to enable secure communications between commercially available mobile devices and Federal Drug Administration (FDA) regulated devices including but not limited to; drug or hormone infusion devices, and sensors to determine a characteristic of a body.

### BACKGROUND OF THE INVENTION

Over the years, bodily characteristics have been determined by obtaining a sample of bodily fluid. For example, diabetics often test for blood glucose levels. Traditional blood glucose determinations have utilized a painful finger prick using a lancet to withdraw a small blood sample. This results in discomfort from the lancet as it contacts nerves in the subcutaneous tissue. The pain of lancing and the cumulative discomfort from multiple needle pricks is a strong reason why patients fail to comply with a medical testing regimen used to determine a change in characteristic over a period of time. Although non-invasive systems have been proposed, or are in development, none to date have been commercialized that are effective and provide accurate results. In addition, all of these systems are designed to provide data at discrete points and do not provide continuous data to show the variations in the characteristic between testing times.

A variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings are useful in monitoring and/or adjusting a treatment regimen which typically includes the regular administration of insulin to the patient. Thus, blood glucose readings improve medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994, which are herein incorporated by reference. Typical thin film sensors are described in commonly assigned U.S. Pat. Nos. 5,390,671; 5,391,250; 5,482,473; and 5,586,553 which are incorporated by reference herein, also see U.S. Pat. No. 5,299,571. However, the wireless controllers or monitors for these continuous sensors provide alarms, updates, trend information and often use sophisticated combination of software and hardware to allow the user to program the controller and/or infusion pump, calibrate the sensor, enter data and view data in the monitor and to provide real-time feedback to the user.

Additionally, the wireless communication between the infusion pump, the controller, and sensor can make the system susceptible to eavesdropping of confidential patient data and potentially hacking attacks to introduce or execute malicious code or commands. Accordingly, security of the wireless communications between the respective system elements is of upmost importance and secondary methods of pairing in addition to commercially available secure pairing methods may be necessary.

### SUMMARY OF THE DISCLOSURE

A monitor system to monitor a characteristic of a user is disclosed. The monitor system includes a medical device having a first machine parsable code, the medical device further having a medical device transmitter, the medical device transmitter configured to broadcast encrypted data indicative of a characteristic of the user. The monitor system further includes a mobile device having a plurality of sensors capable of capturing the first machine parsable code. The mobile device further includes a receiver defined to receive encrypted data broadcast by the medical device. Wherein at least one of the plurality of sensors enables out-of-band pairing between the mobile device and the medical device via the first machine parsable code.

A method to secure wireless communications between a medical device and a controller, is also disclosed. The method includes an operation that initiates at least one of a plurality of sensors associated with the controller. The method further includes an operation that captures a machine parsable code from the medical devices using the initiated sensor. The method then executes program instructions stored on the controller to parse the captured machine parsable code and returns a unique identifier associated with the medical device. The method then executes program instructions stored on the controller to securely pair the controller and the medical device using the unique identifier.

Further disclosed is a method to secure wireless transmission between a wireless device having a machine parsable code and a controller having a plurality of sensors. The method includes an operation that emits the machine parsable code from the wireless device and captures the machine parsable code via one of the plurality of sensors. Also included in the method is an operation that parses the machine parsable code to determine a unique identifier associated with the wireless device. The method also includes an operation that inputs the unique identifier into a pairing application being executed by the controller to securely pair the wireless device and the controller.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIG. 1 is an exemplary illustration of components of a monitor system with secure communications to a mobile device, in accordance with embodiments of the present invention.

FIG. 2 is a block diagram illustrating exemplary basic components within the mobile device, in accordance with embodiments of the present invention.

FIG. 3 is an exemplary block diagram of elements within the medical device, in accordance with embodiments of the present invention.

FIG. 4 is an exemplary block diagram of elements within the analyte sensor, in accordance with embodiments of the present invention.

FIG. 5 is an exemplary illustration of the mobile device in wireless communication with a server network in order to access and install a secure pairing program stored on the server network in accordance with embodiments of the present invention.

FIGS. 6A-6E illustrate imitable screens of the secure pairing program in accordance with embodiments of the present invention.

FIGS. 7A-7D are exemplary machine parsable codes that can be detected by the mobile device, in accordance with embodiments of the present invention.

FIG. 8 is a simplified flow chart that illustrates various operations to securely pair a mobile device and a medical device, in accordance with embodiments of the present invention.

### DETAILED DESCRIPTION

As shown in the drawings for purposes of illustration, the invention is embodied in a monitor system coupled to a subcutaneous implantable analyte sensor set to provide real-time or continuous data recording of the sensor readings for a period of time. In some embodiments the analyte sensor data is transmitted in real-time to a medical device, a mobile device, or both to determine body characteristic data. In another embodiment the analyte sensor data is recorded into memory to be downloaded or transferred to a medical device or mobile device to determine body characteristic data based on the analyte sensor data recorded over the period of time.

In embodiments of the present invention, the analyte sensor set and monitor system are for determining glucose levels in the blood and/or bodily fluids of the user without the use of, or necessity of, complicated monitoring systems that require user training and interaction. However, it will be recognized that further embodiments of the invention may be used to determine the levels of other analytes or agents, characteristics or compositions, such as hormones, cholesterol, medications concentrations, viral loads (e.g., HIV), or the like. In other embodiments, the monitor system may also include the capability to be programmed to record data at specified time intervals. The monitor system and analyte sensor are primarily adapted for use in subcutaneous human tissue. However, still further embodiments may be placed in other types of tissue, such as muscle, lymph, organ tissue, veins, arteries or the like, and used in animal tissue. The analyte sensors may be subcutaneous sensors, transcutaneous sensors, percutaneous sensors, sub-dermal sensors, skin surface sensors, or the like. Furthermore, various embodiments may record sensor readings on an intermittent or continuous basis.

In embodiments that include real-time determination of body characteristic data various types of analysis can be performed by the medical device, mobile device or both on the real-time data. The medical device, being regulated by the Federal Drug Administration, includes various safeguards regarding device security, patient data security, traceability and reporting requirements (e.g., adverse events). As the mobile device may be a mobile smart phone or a customized wireless controller in many embodiments of present invention, safeguarding patient data and data from the sensor during transmission and data manipulation within the mobile device can present a challenge. Establishing trusted secure data transfer between the various elements within the monitor system in conjunction with encryption techniques can provide enhanced data security of sensitive patient data stored on the mobile device.

While the specific embodiments described may be directed toward a mobile device other electronic devices having displays or being connected to displays should also be considered within the scope of this disclosure. For example, televisions capable of running online applications along with networked home gaming consoles while arguably not “mobile,” should be considered within the scope of the disclosure as functioning as the claimed “mobile device”. Additionally, portable gaming devices that are configurable to go online should be considered within the scope of the disclosure.

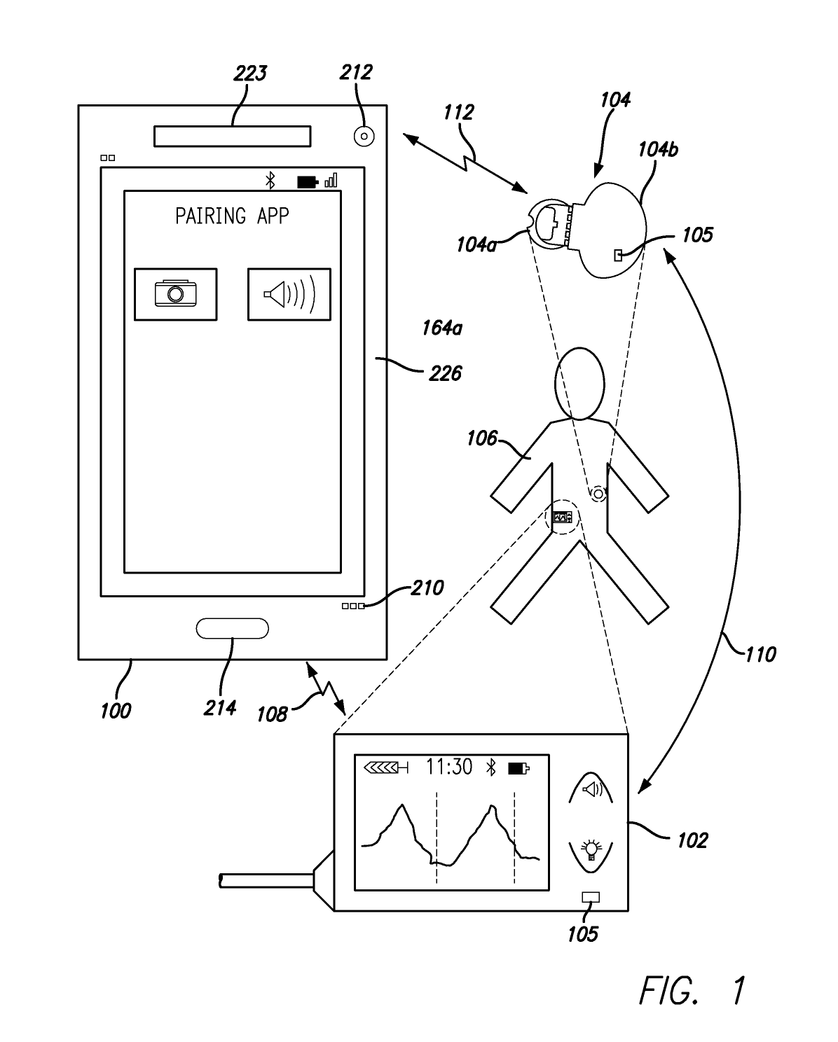

FIG. 1 is an exemplary illustration of components of a monitor system with secure communications to a mobile device 100, in accordance with embodiments of the present invention. Mobile device 100 is shown in wireless communication 108 with medical device 102. Similarly, mobile device 100 is also in wireless communication 112 with analyte sensor set 104 which includes medical sensor 104a that is connected to medical transmitter 104b. As illustrated, medical sensor 104a and medical transmitter 104b are also in wireless communication 110 with medical device 102.

In accordance with some embodiment, the medical device 102 is carried on the person of a user 106 in the manner of an external infusion pump like those commercially available from Medtronic under the trademarked name MINIMED 530G. However, in other embodiments, the medical device 102 can be a different style of infusion pump such as what is commonly referred to as a patch pump attached directly to the user 106. Similarly, in some embodiments the analyte sensor set 104 is attached directly to the skin of the user 106. In one particular embodiment, the analyte sensor set 104 includes two components, the sensor 104a and the transmitter 104b. In such an embodiment the sensor 104a may be attached directly to the user 106 while the transmitter 104b is simply connected to the sensor 104a. This can result in increased comfort and wearability over having both the sensor 104a and transmitter 104b adhered to the skin of the user 106.

In some embodiments the analyte sensor set 104 is a continuous glucose monitoring sensor like those commercially available from Medtronic under the trademarked name ENLITE. However, in other embodiments the analyte sensor set 104 can be configured to measure and broadcast data indicative of a characteristic of the user 106. Similarly, in other embodiments the medical device 102 can be any variety of medical device and form factor as previously discussed. Both the transmitter 104b and the medical device 102 include a machine parsable code 105. Although illustrated on the front of both the medical device 102 and the transmitter 104b in most embodiments the machine parsable code 105 will be discretely placed so as not to be generally visible to passersby. Furthermore, the machine parsable code 105 on the transmitter 104b and the medical device 102 share the same designator for simplicity, in many embodiments every machine parsable code may be parsed into a unique secure code. The specific embodiments discussed above are not intended to be exemplary and should not be construed as limiting the scope of this disclosure.

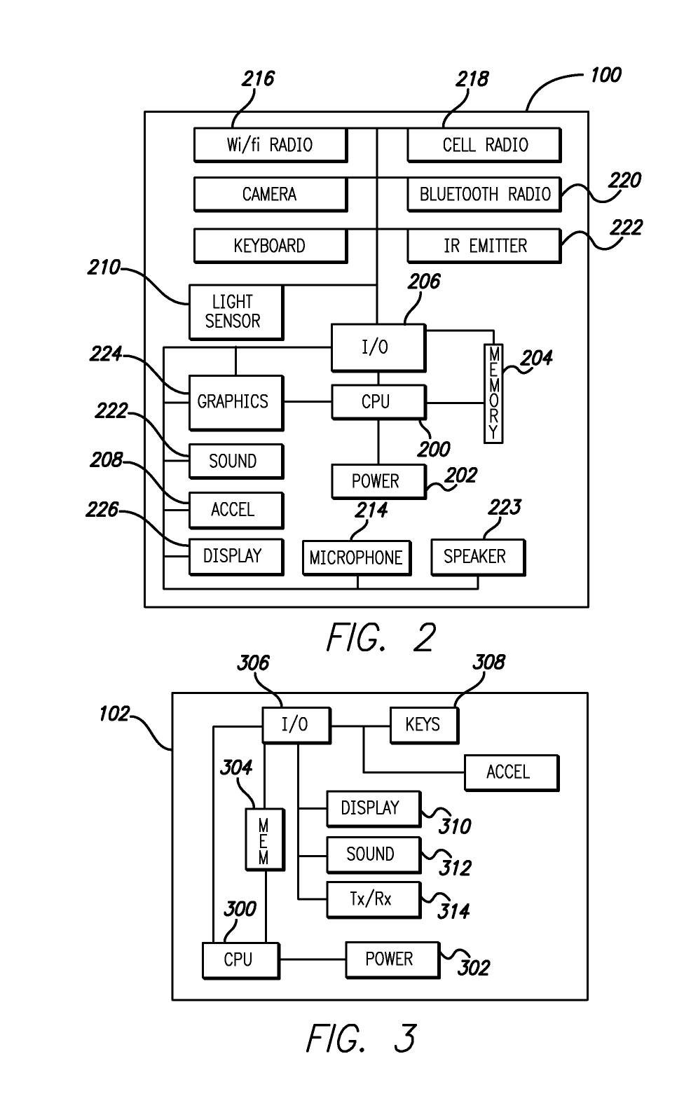

FIG. 2 is a block diagram illustrating exemplary basic components within the mobile device 100, in accordance with embodiments of the present invention. The following description of representational features and components of the mobile device 100 refers to both FIG. 1A and FIG. 2. In the embodiment shown in FIG. 1A the mobile device 100 is intended to resemble a mobile smart phone. However consumer electronics having some or all of the elements discussed in FIG. 2 should be considered within the scope of the disclosure despite the consumer electronic device not necessarily being considered “mobile”. In still other embodiments, the mobile device 100 may further include proprietary or custom controllers for the medical device 102 and/or the analyte sensor 104. The mobile device 100 includes power unit 202 that powers a processor 200 connected to both a memory 204 and an input/output (I/O) controller 206. Power unit 202 may be as simple as a disposable or rechargeable battery while other embodiments may use solar cells, fuel cells, or A/C adapters.

The processor 200 draws power from the power unit 202 and executes program instructions that enable functionality as a smart phone capable of, for example, wireless communications and downloading/executing program instructions for applications or apps. The program instructions executed by the processor 200 can be embedded within the processor 200 (e.g. an on-chip memory cache) while in other embodiments memory 204 stores program instructions along with application data. In still other embodiments program instructions can be stored in both the memory 204 and the processor 200.

The I/O controller 206 being powered by the power unit 202 is coupled to both the processor 200 and the memory 204. In some embodiments the I/O controller monitors a plurality of sensors associated with the mobile device 100. While not a definitive list of potential sensors on the mobile device 100, the mobile device 100 can include, but is not limited to, accelerometers and gyroscopes 208, ambient light sensors 210, digital camera(s) 212 (front facing and/or rear facing), and microphone(s) 214. Also associated with the I/O controller 206 are various radios to enable wireless Wi-Fi (802.11x) 216 communication, various mobile phone radios 218 (EDGE, HSPA, HSPA+, CMDA, CDMA2000, and LTE), Bluetooth radios 220, and IR emitters 222. Other inputs to the mobile device 100 that can be handled via the I/O controller 206 include keyboards 220 (physical or virtual), sound processing 222, and sound output via speakers 223, and graphics 224 that are rendered on a display 226.

FIG. 3 is an exemplary block diagram of elements within the medical device 102, in accordance with embodiments of the present invention. The embodiment of medical device 102 shown in FIG. 1A is an exemplary portable infusion pump while FIG. 3 illustrates basic elements that can be found within any medical device 102 to enable secure communications as contemplated by the present invention. The medical device 102 includes a power supply 302 that provides power to processor 300. Processor 300 is coupled to both memory 304 and I/O controller 306. The processor 300 of the medical device 102 executes program instructions to perform the specific functions of the medical device. The program instructions can be stored in the memory 304 or hardwired into the processor 300. The memory 304 is also coupled to the I/O controller 306. Regarding input, the medical device 102 can include physical keys 308 or a touchscreen. The medical device 102 may also include additional input sensors such as, but not limited to light sensors, accelerometers and cameras. Regarding output, the medical device 102 includes a display 310, audio output 312. The I/O controller 306 also controls a radio 314 to transmit and receive data. In some embodiments the radio 314 operates on the BLUETOOTH standard while in other embodiments the radio 312 operates on the ZIGBEE standard. In particular embodiments the radio 312 is specifically BLUETOOTH LOW ENERGY or BLUETOOTH LE. A benefit of using a standard wireless protocol is that the standard includes data encryption and pairing protocols between devices. The specific embodiments discussed above should not be construed as limiting, rather, the embodiments should be considered illustrative and exemplary.

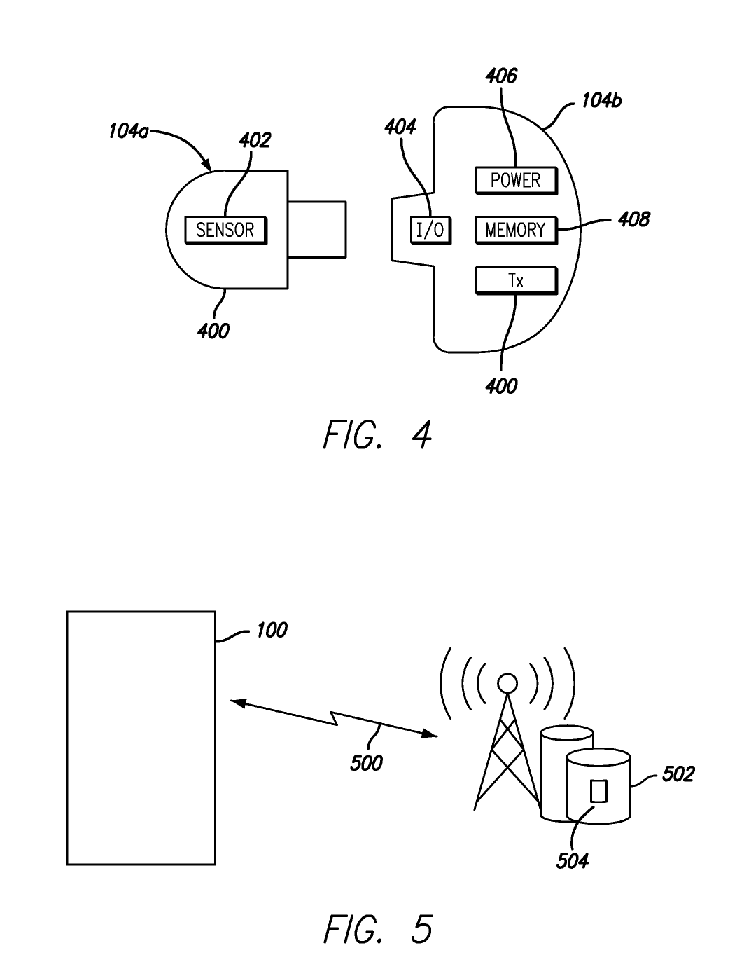

FIG. 4 is an exemplary block diagram of elements within the analyte sensor 104, in accordance with embodiments of the present invention. In some embodiments the analyte sensor 104 includes sensor 104a that includes a carrier 400 that supports the sensor electrodes 402. The carrier 400 assists in the usage of an insertion tool that helps the user insert the senor electrodes at the appropriate depth under their skin. In other embodiments the carrier supports sensors using non-electrode technologies such as but not limited to optical sensors. In the embodiment illustrated in FIG. 4, the sensor 104a couples with the transmitter 104b. The coupling of the sensor 104a and transmitter 104b connects the sensor electrodes 402 to electrical components within the transmitter 104b such as an I/O interface 404, a power supply 406, a memory 408 and a transmitter 410. The I/O interface 404 draws power from the power supply 406 and enables signals from the sensor electrodes 402 to be stored in the memory 408 and transmitted via the transmitter 410 to other devices. In some embodiments the transmitter 410 is one that operates on the BLUETOOTH standard, specifically, the BLUETOOTH LE standard.

The I/O interface 404 can also control optional status indicators on the sensors 104a and/or the transmitter 104b. In one embodiment an LED is used as the status indicator while in other embodiments a small piezo electric sound emitter is the status indicator. In such embodiments, patterns or sequences of LED flashes or audible tones can be used to report on the status if either the sensor 104a or the transmitter 104b. In still other embodiments, the light or sound patterns can assist in secure pairing between the mobile device 100, the medical device 102 and the analyte sensor set 104.

FIG. 5 is an exemplary illustration of the mobile device 100 in wireless communication 500 with a server network 502 in order to access and install a secure pairing program 504 stored on the server network 502 in accordance with embodiments of the present invention. In some embodiments the wireless communication 500 between the mobile device 100 and the server network 502 is similar to the function of the ITUNES or AMAZON APP STORE and the GOOGLE PLAY STORE. As such, a user of the medical device 102 and analyte sensor set 104 would use their mobile device 100 to access the server network 502 (access being either through one of the aforementioned app stores, or via general web browsing) storing the secure pairing program 504. After downloading and installing the secure pairing program can be executed on the mobile device 100.

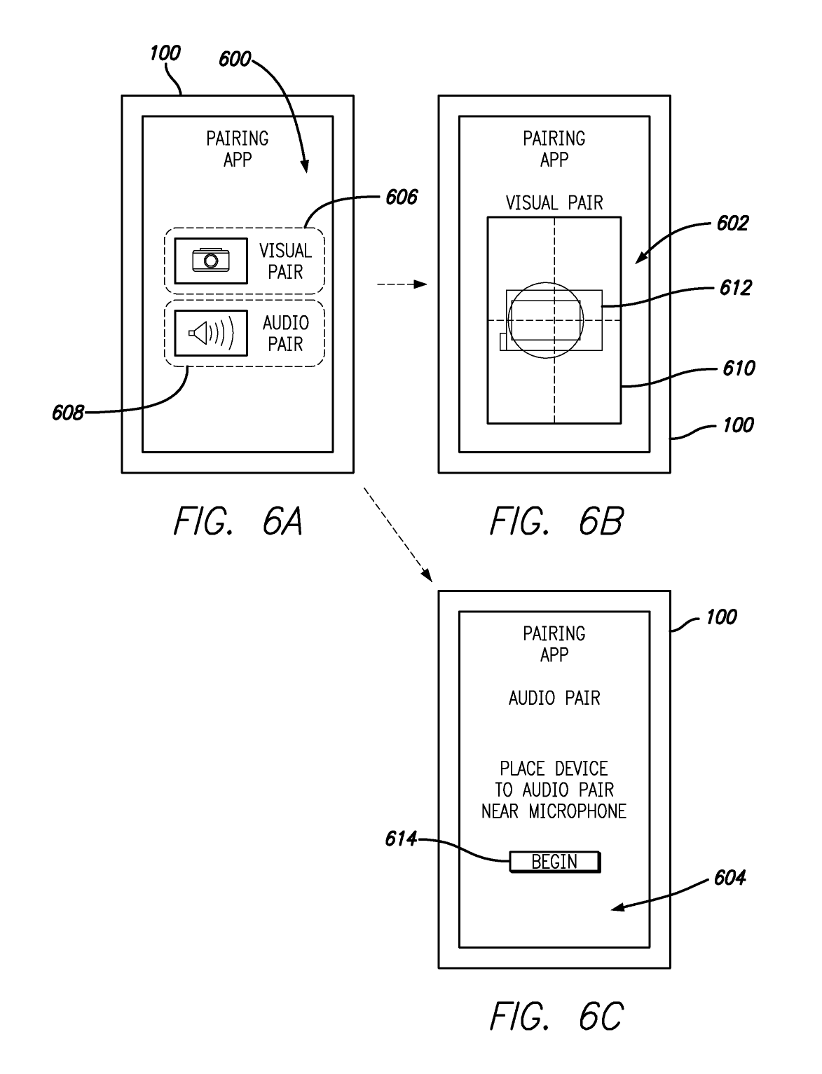

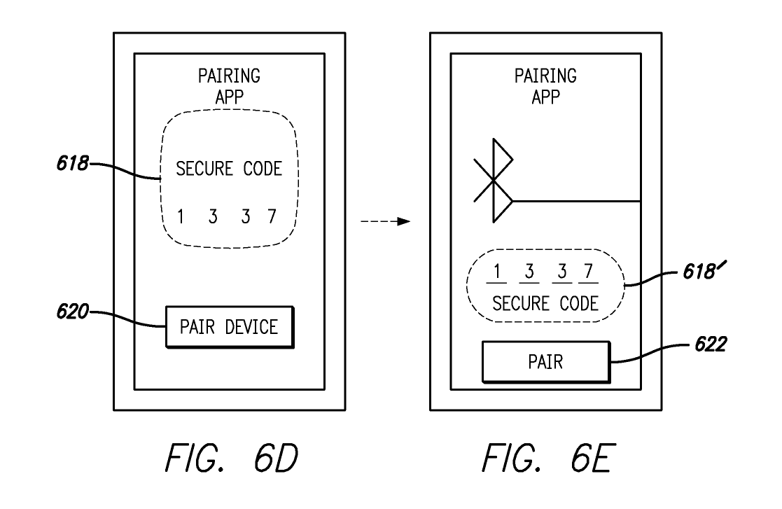

FIGS. 6A-6E illustrate imitable screens of the secure pairing program in accordance with embodiments of the present invention. The embodiment of the secure pairing application discussed in FIGS. 6A-6C includes the option for both visual pairing and audio pairing. In one embodiment, selection of visual pairing 606 results in program instructions being executed to bring up a visual pairing screen 602, as seen in FIG. 6B. In FIG. 6B window 610 within the pairing application shows real-time imagery being taken by a front-facing camera (not shown) integrated into the mobile device. FIG. 6B includes what would be an image 612 of a medical device similar that is behind the mobile device 100. To initiate pairing between the medical device and the mobile device the user would use the mobile device running the pairing application to capture an image of machine parsable code on the medical device.

Referring back to FIG. 6A, selection of audio pairing 608 results in program instructions being executed to bring up audio pairing screen 604 as seen in FIG. 6C. Exemplary audio pairing screen 604 includes instructions to place the device to be paired near a microphone integrated into the mobile device 100. Because mobile devices often have multiple microphones to perform noise cancelling, one embodiment includes an image of a generic medical device being placed near the microphone of a generic telephone receiver. After placing the medical device to be securely paired near the mobile device microphone, the user may be instructed to perform a variety of inputs to prepare the medical device to be paired for pairing. After the medical device to be paired is ready for pairing, the user may press BEGIN 614 on the audio pairing screen 604 along with possible subsequent input to the medical device to initiate the medical device to emit a machine parsable code that is detected by the microphone of the mobile device.

FIG. 6D shows an exemplary screen displaying a secure code 618 after the parsable code is entered in either FIGS. 6B or 6C in accordance with embodiments of the present invention. In one embodiment the pairing application includes program instructions to parse the secure code from the parsable code. In other embodiments, the mobile device parses the parsable code and transmits the parsed code to a server and the server returns to the mobile device the secure code 618. After generating the secure code 618, the user has the option to pair their device with the mobile device by pressing PAIR DEVICE 620.

FIG. 6E is an example of a final pairing screen between a mobile device 100 and a medical device in accordance with embodiments of the present invention. In this embodiment the medical device includes BLUETOOTH pairing that is supplemented with the secure code 618 from FIG. 6D. In some embodiments the BLUETOOTH pairing following the BLUETOOTH standard, specifically BLUETOOTH LOW ENERGY or BLUETOOTH LE. Other embodiments utilize different wireless standards. Selection of PAIR 622 can be accomplished after using the mobile device 100 text entry to enter the secure code 618 and the requisite BLUETOOTH pairing information to complete the secure pairing process.

Other embodiments may only include visual pairing, while still others include only audio pairing. In yet additional embodiments, combinations of visual and audio pairing can be used to establish secure communication. In still other embodiments, other types of pairing can be used such as near field communication (NFC). Further embodiments may include magnetic pulses that can be generated by the medical device that can be detected by a magnetometer within the mobile device. Still other embodiments may utilize other sensors within the mobile phone, such as, but not limited to an accelerometer, ambient light sensors, fingerprint scanners and proximity sensors.

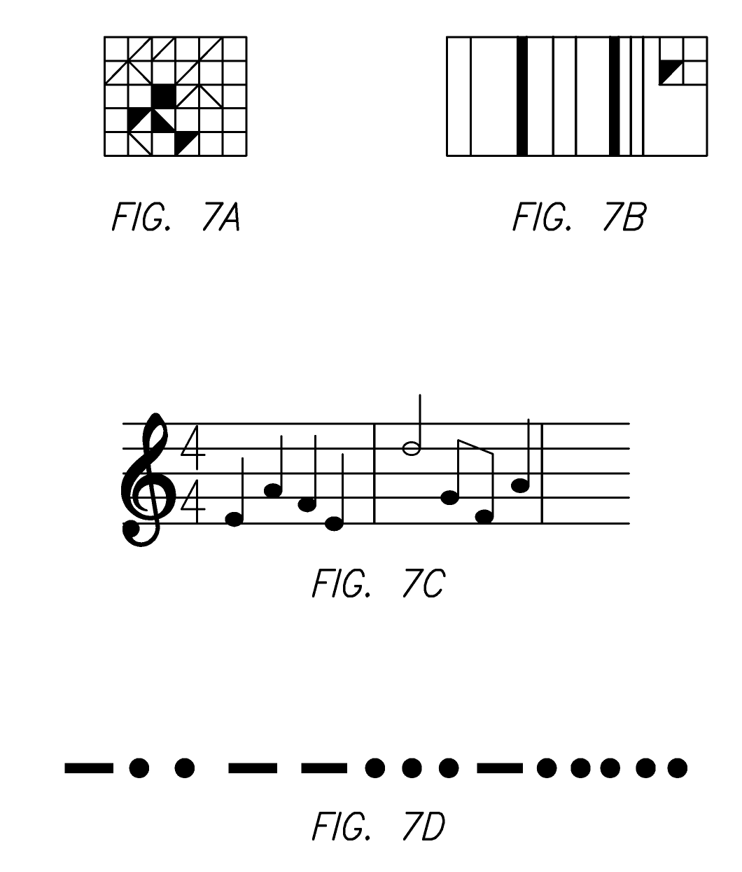

FIGS. 7A-7D are exemplary machine parsable codes that can be detected by the mobile device, in accordance with embodiments of the present invention. FIGS. 7A and 7B are visual machine parsable codes that can be included on a device such a transmitter 104b or a medical device 102 from FIG. 1A. FIGS. 7A and 7B should not be considered limiting regarding the types of graphical machine parsable codes that can be used. Simple barcodes can also be used, along with more complex and/or more simple codes. In some embodiments the machine parsable codes like those in FIGS. 7A and 7B are printer and permanently attached to medical devices. In other embodiments, the machine parsable codes are permanently etched, embossed, engraved or molded into a medical device housing. In embodiments where the medical device includes a screen, the machine parsable code may be displayed on the screen for a preset period of time after the medical device enters a pairing mode.

FIG. 7C is a visual representation of an audio machine parsable code in accordance with embodiments of the present invention. In some embodiments much more complex and lengthy audio codes can be used to generate many more codes. FIG. 7D is a visual representation of still more machine parsable code that can be either audio or visually presented to a mobile device from a medical device. Audio pairing can be completed via if the pattern shown in FIG. 7D is emitted as short audio tones that are detected by the mobile device microphone. Additionally, LED emitters on either the medical device or a transmitter can produce the sequence of light flashes for a camera integrated into the mobile device. For discretion, in some embodiments using an LED emitter an infrared LED is used because it will not be visible to people. In still other embodiments, combinations of sound sequences and light sequences can be used while other embodiments may require the use if sound sequences and visual codes such as those discussed in FIGS. 7A and 7B. In further embodiments, further permutations of different sensors and machine parsable codes can be used to accomplish secure pairing between devices.

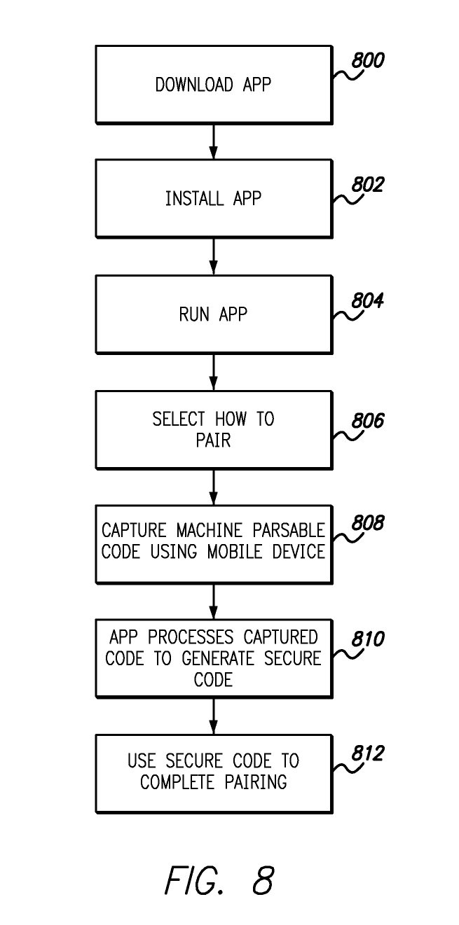

FIG. 8 is a simplified flow chart that illustrates various operations to securely pair a mobile device and a medical device, in accordance with embodiments of the present invention. The flow chart beings with operation 800 where the user downloads a pairing application to the mobile device. In some embodiments the mobile device is a mobile phone and the application is downloaded from a centralized app store such as, but not limited to trademarked applications stores like, APPS FOR WINDOWS, ITUNES, GOOGLE PLAY and AMAZON APP STORE. In other embodiments the mobile device may not be mobile at all, such as a large screen LCD or OLED television set capable of running online applications. In still other embodiments, the mobile device may be a networked game console such as trademarked devices like the PLAYSTATION3, or PS3, XBOX360, PLAYSTATION4, or PS4, XBOX ONE, or WIIU. In still other embodiments, the mobile device may be specialized home monitor device such as the trademarked MYSENTRY remote glucose monitor or the like.

Operation 802 installs the application downloaded in operation 800 onto the mobile device and operation 804 runs the application installed in operation 802. In some embodiments the running of the application results in operation 806 where a user selects how to pair a device with the mobile device. Operation 808 captures the machine parsable code on the medical device using sensors integrated into the mobile device. To securely pair the device sensors integrated with the mobile device are used to capture machine parsable code on the medical device. Although various sensors of the mobile device can be used for pairing one exemplary method is visual pairing using a camera integrated into the mobile device and another exemplary method is audio pairing using a microphone integrated into the mobile device. In instances of visual pairing the medical device would include a machine parsable code that can be captured by the camera. Examples of such machine parsable codes include, but are not limited to pictures, barcodes, and sequences of flashing lights. In instances of audio pairing, the medical device would include a sound emitting device that would be placed in functional proximity to a microphone integrated into the mobile device. The audio machine parsable code could include, but is not limited to multi tonal sound sequences and tonal pulses.

Operation 810 executes program instructions within the application to process the machine parsable code captured in operation 808 into a secure code. In one embodiment the entirety of the processing is performed on the mobile device. In this embodiment, the application downloaded and install on the mobile device includes the ability to parse values for captured machine parsable code. In other embodiments, the mobile device requires an internet connection or internet access to query a secure database and have the secure database return a secure code. In operation 812 the secure code generated in operation 810 is used to complete the secure pairing process.

While the description above refers to particular embodiments of the present invention, it will be understood that many modifications may be made without departing from the spirit thereof. The accompanying claims are intended to cover such modifications as would fall within the true scope and spirit of the present invention. For example, specific embodiments were disclosed regarding secure communication within a personal area network (PAN) that includes a medical device such as an infusion pump, a mobile device such as a smart phone or custom controller, and analyte sensor set. However, a personal area network that includes only two devices such as a medical device and mobile device, or medical device and analyte sensor set, or mobile device and analyte sensor set should be considered within the scope of this disclosure if appropriate hardware and software is included in each respective device to enable secure pairing. Similarly, the scope of this disclosure should not be construed to be restricted to personal area networks within the medical device industry. The embodiments and techniques disclosed should be construed to be adaptable to any environments that can benefit from secure wireless communication within a wide area network, local area network or personal area network should the appropriate hardware and software is included with the devices to be securely paired.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A monitor system to monitor a characteristic of a user, the system comprising:
a medical device having a first machine parsable code, the medical device further having a medical device transmitter, the medical device transmitter configured to broadcast encrypted data indicative of a characteristic of the user; and
a mobile device having a plurality of sensors capable of capturing the first machine parsable code, the mobile device further includes a receiver defined to receive encrypted data broadcast by the medical device,
wherein at least one of the plurality of sensors enables out-of-band pairing between the mobile device and the medical device via the first machine parsable code.

2. A monitor system as described in claim 1, further including,
a medical sensor having a sensor transmitter coupled to a medical sensor power source, the medical sensor broadcasting encrypted data indicative of glucose concentration within the user,
wherein the medical device is a portable insulin pump further having a pump receiver defined to receive wireless communications from the medical sensor.

3. A monitor system as described in claim 2, further including
a second machine parsable code being located on the medical sensor, the second machine parsable code enabling out-of-band pairing between the medical sensor and the mobile device.

4. A monitor system as described in claim 2, wherein the mobile device executes program instructions for an application, the program instructions defined to parse a value encoded in the first machine parsable code, the value being a unique identifier that enables pairing between the mobile device and the medical device via the application.

5. A monitor system as described in claim 4, wherein the mobile device further includes internet access, the internet access transmitting the parsed value to a secure database, the secure database returning a database unique identifier that enables pairing between the mobile device and the medical device via the application.

6. A monitor system as described in claim 3, wherein the mobile device executes program instructions for an application, the program instructions defined to parse a value encoded in the second machine parsable code, the value being a unique identifier that enables pairing between the medical sensor and the mobile device via the application.

7. A monitor system as described in claim 6, wherein the mobile device further includes mobile data access, the mobile data access transmitting the parsed value to a secure database, the secure database returning a secure code that enable pairing between the mobile device and the medical sensor via the application.

8. A monitor system as described in claim 1, wherein the first machine parsable code is an image and one of the plurality of sensors of the mobile device is a camera.

9. A monitor system as described in claim 1, wherein the first machine parsable code is a series of audio tones and one of the plurality of sensors of the mobile device is a microphone.

10. A monitor system as described in claim 1, wherein the first machine parsable code is a sequence of flashing lights emitted by an LED and one of the plurality of sensors of the mobile device is a camera defined to capture the sequence of flashing lights.

11. A method to secure wireless communications between a medical device and a controller, the method comprising:
initiating at least one of a plurality of sensors associated with the controller;
capturing a machine parsable code from the medical devices using the initiated sensor;
executing program instructions stored on the controller to parse the captured machine parsable code;
returning a unique identifier associated with the medical device;
executing program instructions stored on the controller to securely pair the controller and the medical device using the unique identifier.

12. A method as described in claim 11, wherein the machine parsable code is an image and the one of the plurality of sensors of the controller is a camera.

13. A method as described in claim 11, wherein the machine parsable code is a sequence of light flashes and one of the plurality of sensors of the controller is a camera.

14. A method as described in claim 11, wherein the machine parsable code is a sequence of different audible tones and one of the plurality of sensors is a microphone.

15. A method as described in claim 11, wherein the unique identifier is returned after executing program instructions on the controller to parse the machine parsable code.

16. A method as described in claim 11, wherein the parsed machine parsable code is transmitted to a remote database and the remote database returns the unique identifier.

17. A method to secure wireless transmissions between a wireless device having a machine parsable code and a controller having a plurality of sensors, the method comprising:
emitting the machine parsable code from the wireless device;
capturing the machine parsable code via one of the plurality of sensors;
parsing the machine parsable code to determine a unique identifier associated with the wireless device;
inputting the unique identifier into a pairing application being executed by the controller to securely pair the wireless device and the controller.

18. A method as described in claim 17, wherein the emitted machine parsable code is a sequence of light flashes and the one of the plurality of sensors is a camera.

19. A method as described in claim 17, wherein the emitted machine parsable code is a sequence of audible tones and the one of the plurality of sensors is a microphone.

20. A method as described in claim 17, wherein the emitted machine parsable code is combination of a sequence of light flashes and a sequence of audible tones and the plurality of sensors includes a camera and a microphone.
